(19) United States
(12) Patent Application Publication
(10) Pub. No.: US 2020/0000568 A1
(43) Pub. Date: Jan. 2, 2020

(54) PERIODONTAL POCKET EXAMINATION APPARATUS

(71) Applicant: TANITA CORPORATION, Tokyo (JP)

(72) Inventor: Mikio SHINDO, Tokyo (JP)

(21) Appl. No.: 16/570,161

(22) Filed: Sep. 13, 2019

Related U.S. Application Data
(63) Continuation of application No. PCT/JP2018/005381, filed on Feb. 16, 2018.

(30) Foreign Application Priority Data
Mar. 15, 2017 (JP) 2017-049420

(51) Int. Cl. A61C 19/04; A61B 1/06; A61B 1/24; A61B 1/00; G02F 1/29; G01B 9/02

(52) U.S. Cl. CPC A61C 19/043; A61B 1/06; A61B 1/24; A61B 1/00096; A61B 1/00039; G01B 9/02091; G01B 9/0203; G01B 9/0205; G02F 1/29

(57) ABSTRACT

An examination probe used in an apparatus for examining the depth of a periodontal pocket includes a light-emitting portion and a gripping portion. The light-emitting portion includes a crystal deflecting element, which deflects, in a specific direction, measuring light split off from low-interference light, and an f-θ lens for rendering parallel the measuring light that has been deflected. Measuring light that has been rendered parallel such as a measuring light beam is emitted from an opening of the light-emitting portion and irradiates a gum or tooth of a subject. An optical tomographic image of the gum or tooth is generated from interference signals obtained based on reflected light, allowing the depth of a periodontal pocket to be determined. The light-emitting portion protrudes further than the gripping portion does in the direction of emission of the measuring light such as the measuring light beam.

### TECHNICAL FIELD

This invention relates to a periodontal pocket examination apparatus.

### BACKGROUND ART

Measurement of the depth of a periodontal pocket is carried out as one example of an examination of periodontal disease. In general, the depth of a periodontal pocket is measured visually as by a dentist inserting a rod-like measuring instrument referred to as a “pocket probe” into the periodontal pocket. However, there are occasions where the result of measurement it not necessarily accurate owing to the extent of the ability of the dentist or the like, the angle of insertion of the pocket probe and visual error, etc. Further, there is concern that, owing to bleeding from the gums at the time of examination, affected parts free of periodontal disease will become infected with periodontal disease. For these reasons, consideration has been given to the measurement of periodontal pocket depth non-invasively using an optical coherence tomographic diagnostic apparatus (Patent Documents 1, 2).

### PRIOR-ART DOCUMENTS

### Patent Documents

Patent Document 1: Japanese Patent Application Laid-Open No. 2009-131313
Patent Document 2: Japanese Patent Application Laid-Open No. 2009-148337

In order to measure periodontal pocket depth using an optical interference tomographic diagnostic apparatus, miniaturization is required because it is necessary to insert an examination probe into the oral cavity of the patient. However, the inventors have recognized that, with the art described in Patent Documents 1 and 2, the examination probe itself is large in size owing to use of a galvanomirror. The inventors have further recognized that it is difficult to assure accuracy of measurement owing to noise ascribable to vibration or the like produced when the driving system of the galvanomirror is driven. The inventors have further recognized that it is desirable to improve the operability of the examination probe because it is preferred that the measuring light be emitted perpendicular to the depth direction of the periodontal pocket in order to measure the depth of the periodontal pocket accurately.

### DISCLOSURE OF THE INVENTION

An object of the present invention is to improve the operability of an examination probe while miniaturizing the examination probe.

A periodontal pocket examination apparatus according to the present invention is characterized by comprising: an optical divider for splitting low-interference light into measuring light and reference light; a crystal deflecting device on which the measuring light split off by said optical divider is incident for deflecting the incident measuring light in a specific direction (or on the side of a specific direction) in accordance with an applied voltage and for emitting the measuring light deflected; a parallelizing device for aligning into parallel light the measuring light emitted from said crystal deflecting device; a photodetector for detecting reflected light and outputting an interference signal, the reflected light being reflected measuring light which is reflected from a gum or tooth owing to irradiation of the gum or tooth with the measuring light aligned parallel by said parallelizing device and reflected reference light which is split off by said optical divider and reflected by a reference surface; periodontal pocket data generator (processor) for generating data regarding depth of a periodontal pocket based on the interference signal output from said photodetector; and an examination probe including said crystal deflecting device, said parallelizing device and a gripping portion, the gripping portion extends from one side face of a light-emitter for emitting from an opening the measuring light aligned parallel by said parallelizing device, said light-emitter portion protruding further than said gripping portion does in the direction of emission of the measuring light.

The light-emitter of the examination probe may be adapted so as to be freely deformable such that the light-emitter of the examination probe is deformed in a case where a force is applied to the light-emitter of the examination probe in the direction opposite the direction of emission of the measuring light, and returns to the shape thereof that prevailed prior to deformation in a case where the force applied to the light-emitter of the examination probe is released.

The examination probe may be freely deformable such that the light-emitter of the examination probe, at an upper portion and lower portion of the opening of the light-emitter, is deformed in a case where a force is applied, over at least a portion in the width direction, in the direction opposite the direction of emission of the measuring light, and returns to the shape thereof that prevailed prior to deformation in a case where the force applied to the light-emitter of the examination probe is released.

At least a part of the upper portion and lower portion is constituted by an elastic member such that a front face of the light-emitter is capable of being brought into close contact with a gum or tooth.

The apparatus further comprises an angle sensor for detecting at least one among roll angle, pitch angle and yaw angle of the examination probe.

By way of example, the crystal deflecting device deflects the incident measuring light in such a manner that deflection width of the measuring light emitted from the light-emitter of the examination probe is enough deflection width for measurement of depth of a periodontal pocket in a single scan.

By way of example, the periodontal pocket data generator generates data regarding depth of a periodontal pocket, based on interference signals output from the photodetector by using the examination probe to perform measurement at least at two locations at positions that differ in height, in a case where the deflection width of the measuring light emitted from the light-emitter of the examination probe is less than enough deflection width for measurement of depth of a periodontal pocket in a single scan.

The apparatus further comprises optical tomographic image generator for generating at least two optical tomographic images, based on interference signals output from the photodetector by using the examination probe to perform measurement at least at two locations at positions that differ in height, in a case where the deflection width of the measuring light emitted from the light-emitter of the examination probe is less than enough deflection width for measurement of depth of a periodontal pocket in a single scan. In this case, by way of example, the periodontal pocket data generator generates data regarding depth of a periodontal pocket by combining and processing at least two optical tomographic images generated by the optical tomographic image generator.

Preferably, a position corresponding to the light-emission position of the measuring light emitted from the light-emitter is marked on the exterior of the light-emitter with the exception of the front face thereof.

By way of example, the opening of the examination probe or the front face of the light-emitter of the examination probe has the shape of a square, a circle, a rectangle whose side in the vertical direction is shorter than the side in the longitudinal direction, or an ellipse the longitudinal direction of which is the major axis and the vertical direction of which is the minor axis.

The gripping portion includes a neck portion and a base-end portion and, in a case where the base-end portion extends from one side face of the light-emitter of the examination probe via the neck portion, the neck portion curves in the direction opposite the direction of the light emission and protrudes in the direction opposite the direction of the light emission, or the light-emitter extends further than the neck portion does in the direction of the light emission, or one end of the neck portion is secured to a rear end of the light-emitter on one side face thereof and the other end of the neck portion protrudes further than the one end of the neck portion does in the direction of the light emission, or at least one of an upper-end portion and lower-end portion of the neck portion is cut away, by way of example.

For example, the examination probe is such that a straight line in the longitudinal direction along which the gripping portion of the examination probe extends and a straight line in the direction of the measuring light prior to deflection thereof by the crystal deflecting device are non-parallel.

For example, the examination probe is such that a straight line in the longitudinal direction along which the gripping portion of the examination probe extends and a straight line in the direction of the measuring light prior to deflection thereof by the crystal deflecting device may be orthogonal.

The apparatus may further comprise a voltage circuit for impressing the above-mentioned applied voltage upon the crystal deflecting device. In this case, it is preferred that, on the one hand, when the applied voltage impressed by the voltage circuit is a positive voltage, the crystal deflecting device deflects the measuring light more in the specific direction in response to an increase in the positive voltage, and when the applied voltage impressed by the voltage circuit is a negative voltage, the crystal deflecting device deflects the measuring light more in the direction opposite the specific direction in response to an increase in the negative voltage.

The light-emitter of the examination probe may have a transparent plate. In this case, it is preferred that the transparent plate be fixed at a position inwardly of the opening of the light-emitter in the direction opposite the direction of the light emission.

In accordance with the present invention, since the measuring light is deflected by the crystal deflecting device, the examination probe can be miniaturized in comparison with a case where the measuring light is deflected using a galvanomirror that requires a driving unit. Further, in the examination probe, the light-emitter that emits the parallelized measuring light from the opening protrudes further along the direction of emission of the measuring light than the gripping portion does. Therefore, in a case where the user such as a dentist inserts the examination probe into the oral cavity of the subject of measurement such as a patient by gripping the gripping portion, the fingers holding the gripping portion can be prevented from contacting the teeth, etc., of the subject, and operability of the examination probe can be improved as well.

### BRIEF DESCRIPTION OF THE DRAWINGS

FIG. 1 is a block diagram illustrating the construction of a periodontal pocket examination apparatus;

FIG. 2 illustrates the manner in which a gum and a tooth are irradiated with measuring light;

FIG. 3 illustrates the manner in which measuring light is deflected;

FIG. 4 is a perspective view of an examination probe;

FIG. 5 illustrates the manner in which a gum and a tooth are irradiated with measuring light;

FIG. 6A to FIG. 6E are examples of interference signals;

FIG. 7 is an example of optical tomographic images of a gum and a tooth;

FIG. 8 is an example of measuring light deflected by a crystal deflecting element;

FIG. 9 illustrates the manner in which a gum and a tooth are irradiated with measuring light;

FIG. 10A and FIG. 10B are examples of interference signals;

FIG. 11A is a perspective view of an examination probe and FIG. 11B is a sectional view as seen along line XIB-XIB of FIG. 11A;

FIG. 12A is a perspective view of an examination probe and FIG. 12B is a sectional view as seen along line XIIB-XIIB of FIG. 12A;

FIG. 13A and FIG. 13B are perspective views of an examination probe;

FIG. 14A illustrates the manner in which a roll angle is detected, FIG. 14B the manner in which a yaw angle is detected, and FIG. 14C the manner in which a pitch angle is detected; and

FIG. 15A through FIG. 15D are perspective views of examination probes.

### BEST MODE FOR CARRYING OUT THE INVENTION

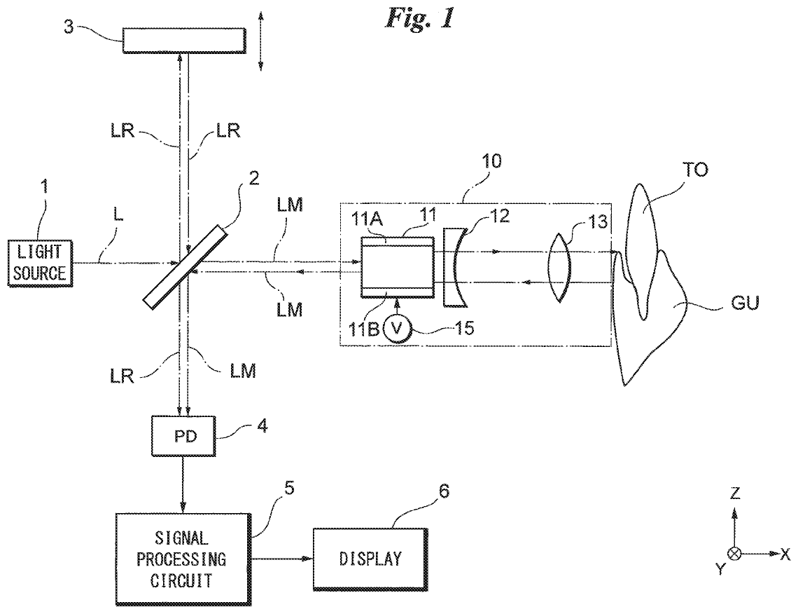

FIG. 1, which illustrates an embodiment of the present invention, is a block diagram showing the construction of a periodontal pocket examination apparatus.

Low-interference light (low-coherence light) L is emitted from a light source 1 such as an SLD (Super Luminescent Diode). The low-interference light L is split into measuring light LM and reference light LR by a beam splitter (optical divider) 2. It will suffice if low-interference light L is emitted from the light source 1, and use may be made of another light source such as a gas laser, semiconductor laser or laser diode.

The measuring light LM split off by the beam splitter 2 impinges upon an examination probe 10. The examination probe 10 includes a crystal deflecting element (a crystal deflecting device) 11, a concave lens 12 and an f-θ lens 13. (Although the f-θ lens corresponds to a parallelizing element, another element will suffice if it is capable of rendering parallel the light emitted from the crystal deflecting element 11.)

The measuring light LM incident upon the examination probe 10 impinges upon the crystal deflecting element 11. An electrode 11A is formed on the upper surface of the crystal deflecting element 11, and an electrode 11B is formed on the lower surface of the crystal deflecting element 11. When a voltage from a voltage circuit 15 is applied to the electrodes 11A and 11B, the crystal deflecting element 11 deflects and emits the incident measuring light LM in accordance with the applied voltage in such a manner that the light after deflection is emitted in a specific direction. (It will suffice if the light after deflection is emitted on the side of a specific direction, not in a specific direction.) The “specific direction (or “on the side of a specific direction”) refers to a direction orthogonal to the direction of the measuring light prior to its deflection. In FIG. 1, if we let the left-right direction, the direction perpendicular to the plane of the drawing and the vertical direction be the X-axis, Y-axis and Z-axis, respectively, then the direction of the measuring light LM that impinges upon the crystal deflecting element 11 will be the positive direction along the X-axis, and the specific direction along which the measuring light is deflected by the crystal deflecting element 11 will be any direction in the plane of the X- and Z-axes. In this embodiment it is assumed that the measuring light is deflected along the positive and negative directions of the Z-axis. The specific direction taken on by the light after its deflection is not limited to a direction along which the light after deflection will be parallel to a specific direction; it will suffice if the light after deflection is deflected even slightly in a specific direction. For example, if the measuring light LM traveling along the positive direction of the X-axis is deflected at a deflection angle of 90 degrees (in actuality, if the deflection angle were to be 90 degrees, the measuring light LM after deflection would not irradiate the gum or tooth that is the object of measurement; hence, the deflection angle would be greater than −90 degrees and less than 90 degrees), the measuring light will be deflected in a direction parallel to the Z-axis. However, deflection is not limited to such case, for it will suffice if the measuring light LM is deflected so as to lean more in the direction indicated by the Z-axis than by the X-axis even if the deflection angle is less than 90 degrees (even if the deflection angle is 1 degree).

The crystal deflecting element 11 refers to an element that applies a voltage to a crystal and deflects incident light in accordance with the applied voltage, and use can be made of either an acousto-optic element that deflects incident light by the acousto-optic effect, or an electro-optic element that deflects incident light by the electro-optic effect. An example of an acousto-optic element is one utilizing a crystal such as dihydrogenide glass or quartz, and an example of the electro-optic element is one utilizing KTN crystal, which is an oxide crystal consisting of calcium (K), tantalum (Ta) and niobium (Nb), or a barium borate crystal. The light deflecting effect of KTN crystal affects the deflection component in the direction of the internal electric field. Accordingly, in a case where KTN crystal is utilized as the crystal deflecting element 11, the direction of deflection of the low-interference light emitted by the light source 1 and the direction of the electric field produced by the voltage impressed upon the KTN crystal are stipulated in such a manner that the direction of the electric field produced by the voltage impressed upon the KTN crystal and the direction of deflection of the low-interference light emitted by the light source 1 will coincide. In this embodiment, it is assumed that KTN crystal is utilized as the crystal deflecting element 11.

The measuring light LM deflected by the crystal deflecting element 11 impinges upon the concave lens 12. Since the KTN crystal itself has the function of a convex lens, a convex lens effect happens to be produced. The concave lens 12 is provided in order to cancel out the convex lens effect.

The measuring light LM transmitted through the concave lens 12 is rendered parallel by the f-θ lens 13 (parallelizing element) and irradiates a gum GU and a tooth TO which are to undergo measurement. The measuring light LM reflected from the gum GU and tooth TO passes through the interior of the examination probe 10, is reflected in the beam splitter 2 and impinges upon a photodiode 4 (photodetector).

Further, the reference light LR split off in the beam splitter 2 is reflected at a reference mirror 3 (reference surface) freely movable in the direction of propagation of the reference light LR and in the direction opposite thereto (along the positive and negative directions of the Z-axis in the embodiment shown in FIG. 1). The reflected reference light LR passes through the beam splitter 2 and impinges upon the photodiode 4.

When, by moving the reference mirror 3, equality is established between a propagation distance, which is the sum total of propagation distance traveled until the measuring light LM irradiates the gum GU and tooth TO undergoing examination and propagation distance traveled until light reflected from the gum GU and tooth TO undergoing examination impinges upon the photodiode 4, and a propagation distance, which is the sum total of propagation distance traveled until the reference light LR irradiates the reference mirror 3 and light reflected from the reference mirror 3 impinges upon the photodiode 4, interference occurs between the measuring light LM and reference light LR and the photodiode 4 outputs an interference signal.

The interference signal output from the photodiode 4 is input to a signal processing circuit 5 (periodontal pocket data generator, processor), and signals representing optical tomographic images of the gum GU and tooth TO (data regarding the depth of a periodontal pocket) are generated. By inputting the generated signals representing the optical tomographic images to a display unit 6, the optical tomographic images of the gum GU and tooth TO are displayed on the display screen of the display unit 6. Processing for extracting the contours of the optical tomographic images is executed in the signal processing circuit 5, whereby the depth of a periodontal pocket between the gum GU and tooth TO is calculated. The calculated depth of the periodontal pocket also is displayed on the display screen of the display unit 6. Although optical tomographic images are generated and the depth of the periodontal pocket is calculated from the generated optical tomographic images, an arrangement may be adopted in which, rather than generate optical tomographic images, numerical data representing the depth of the periodontal pocket (such numerical data also is considered to be data regarding the depth of the periodontal pocket) is calculated in the signal processing circuit 5 and the depth of the periodontal pocket is displayed on the display screen of the display unit 6.

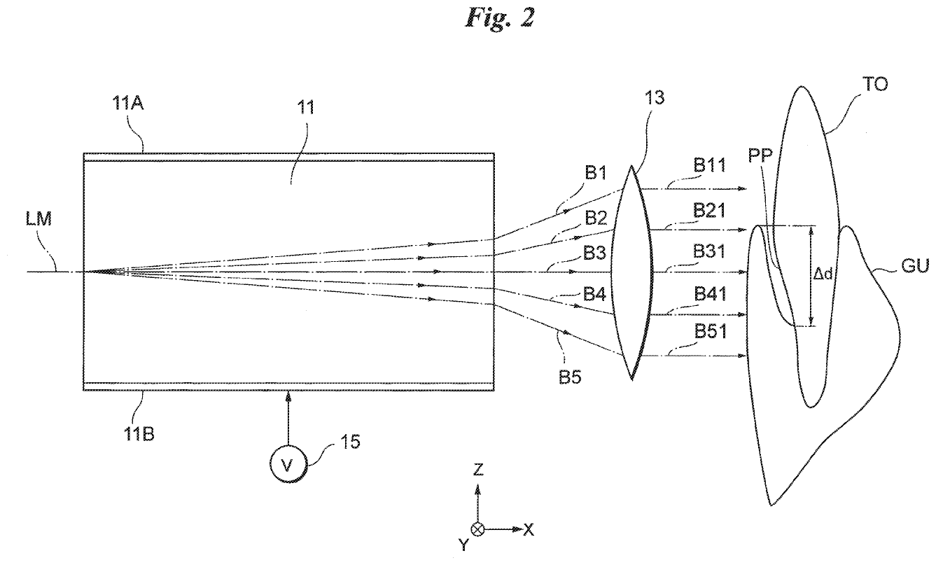

FIG. 2 illustrates the manner in which the measuring light LM is deflected by the crystal deflecting element 11. In FIG. 2, the concave lens 12 is omitted.

When voltage is being applied to the crystal deflecting element 11 by voltage circuit 15, the measuring light LM incident upon the crystal deflecting element 11 is deflected. The deflection angle of the measuring light LM in the crystal deflecting element 11 differs depending upon the voltage applied to the crystal deflecting element 11; the higher the voltage, the more the measuring light is deflected. For example, by application of a positive voltage, the measuring light LM is deflected along the positive direction of the Z-axis, as indicated by symbols B1 (measuring light beam B1). If there is applied a positive voltage smaller than the positive voltage which is in effect in the case where a measuring light beam B1 is obtained, then the measuring light LM will be deflected at a deflection angle, which is smaller than that of the measuring light beam B1, along the positive direction of the Z-axis, as indicated by symbols B2 (measuring light beam B2). If there is no applied voltage, the measuring light LM will not be deflected, as indicated by symbols B3 (measuring light beam B3). Furthermore, by making the applied voltage negative, the measuring light LM is deflected along the negative direction of the Z-axis, as indicated by symbols B5 (measuring light beam B5). If there is applied a negative voltage smaller than that in effect in the case where the measuring light beam B5 is obtained, then the measuring light LM will be deflected at a deflection angle, which is smaller than that of the measuring light beam B5, along the negative direction of the Z-axis, as indicated by symbols B4 (measuring light beam B4). It goes without saying that, although there are an infinite number of measuring light beams obtained by deflection using the crystal deflecting element 11, five measuring light beams B1 to B5 are illustrated in order to facilitate understanding.

Thus, owing to the crystal deflecting element 11, the measuring light after deflection has a deflection in a specific direction (in FIG. 2, the positive direction and negative direction along the Z-axis, these being directions orthogonal to the direction of the X-axis, which is the direction of the measuring light LM prior to the deflection thereof), as in the manner of the measuring light beams B1 to B5 shown in FIG. 2. Further, deflection of the measuring light by the crystal deflecting element 11 can also be performed such that the measuring light LM before deflection and the measuring light beams B1 to B5 after deflection will all lie along the same plane.

The measuring light beams B1 to B5 are rendered parallel by the f-θ lens 13 so as to be made parallel to the measuring light LM that prevailed prior to its deflection by the crystal deflecting element 11. The thus parallelized measuring light beams B11, B21, B31, B41 and B51 irradiate the gum GU and tooth TO undergoing examination. (The measuring light beams B11, B21, B31, B41 and B51 do not necessarily irradiate both the gum GU and the tooth TO; depending upon the irradiated position, there is a measuring light beam which irradiates the tooth TO but not the gum GU. For example, since the gum GU is not present at the position irradiated with the measuring light beam B11, the measuring light beam B11 irradiates the tooth TO but not the gum GU). Depth Δd of a periodontal pocket PP is calculated based upon the reflected light beams. As shown in FIG. 2, the measuring light beams deflected by the crystal deflecting element 11 are indicated by the symbols B1, B2, B3, B4 and B5, and the measuring light beams rendered parallel by the f-θ lens 13 are indicated by the symbols B11, B21, B31, B41 and B51.

In FIG. 2, the deflection angle is enlarged by enlarging the voltage applied to the crystal deflecting element 11. However, as an example of a technique for improving the angular range over which deflection is possible, it is also possible to enlarge the length of the crystal deflecting element 11 (length along the direction of the X-axis, which is the direction traversed by the measuring light LM).

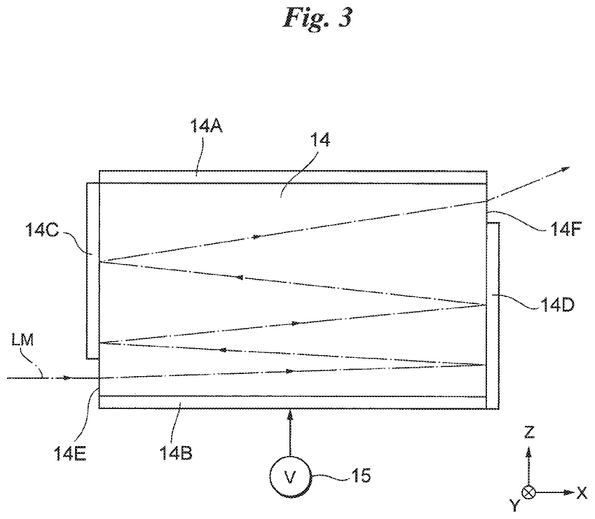

FIG. 3 illustrates a modification of crystal deflecting element 14.

In the crystal deflecting element 14 shown in FIG. 3, a first reflecting mirror 14C is formed on the surface on which the measuring light LM is incident, and a second reflecting mirror 14D is formed on the surface from which the measuring light LM is emitted. The lower end of the light-incident surface (the surface on the left side in FIG. 3) of the crystal deflecting element 14 does not have the first reflecting mirror 14C formed thereon and serves as a window 14E. The upper end of the light-emission surface (the surface on the right side in FIG. 3) of the crystal deflecting element 14 does not have the second reflecting mirror 14D formed thereon and serves as a window 14F.

When a voltage is applied to electrodes 14A and 14B formed respectively on upper and lower surfaces of the crystal deflecting element 11, the measuring light LM that has impinged upon the crystal deflecting element 14 from the window 14E of the light-incident surface is deflected inside the crystal deflecting element 14 and is then reflected by the second reflecting mirror 14D formed on the light-emission surface. The measuring light LM reflected by the second reflecting mirror 14D is deflected inside the crystal deflecting element 14 and is then reflected by the first reflecting mirror 14C formed on the light-incident surface. While reflection by the first reflecting mirror 14C and second reflecting mirror 14D is thus repeated, the light is deflected and emitted from the window 14F on the light-emission side. Since the propagation distance within the crystal deflecting element 14 is lengthened, the deflection angle increases.

It goes without saying that the deflection angle is changed by changing the applied voltage also in the crystal deflecting element 14 shown in FIG. 3.

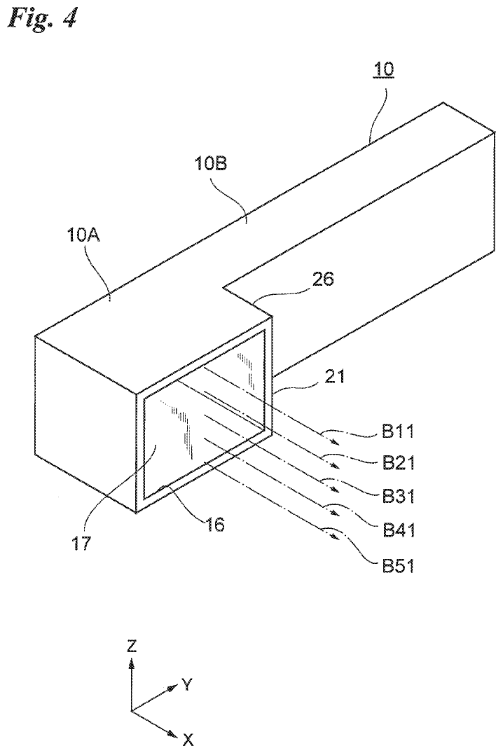

FIG. 4 is a perspective view as seen from the front of the examination probe 10.

As set forth above, the examination probe 10 includes the crystal deflecting element 11, the concave lens 12 and the f-θ lens 13. However, rather than the concave lens 12 being provided, the f-θ lens 13 may be a lens such as an aspherical lens that has the function of the concave lens 12. That is, it will suffice if the arrangement is such that the concave lens 12 and f-θ lens 13 correct the characteristic of the crystal deflecting element 11 and parallel light beams are obtained.

The examination probe 10 includes a light-emitting portion (a light-emitter) 10A and a gripping portion 10B. The gripping portion 10B extends from the right-side face 26 (one side face) of the light-emitting portion 10A.

An opening 16 is formed on a front face 21 of the light-emitting portion 10A (on the side from which the measuring light beams B11 to B51 are emitted, as described with reference to FIG. 2). A transparent plate 17 is fitted into the opening 16. The opening 16 is a rectangle in shape as seen from the front (the side of the front face 21 in FIG. 4 is taken as the front), where the side in the vertical direction (the direction along the Z-axis) is shorter than the side in the longitudinal direction (the direction almond the Y-axis). The measuring light beams B1 to B5, which have been deflected and rendered parallel, are emitted from the opening 16.

The light-emitting portion 10A protrudes from the gripping portion 10B along the direction of emission of the measuring light beams B1 to B5 (the positive direction along the X-axis). Therefore, even if the user such as a dentist grips the gripping portion 10B, inserts the examination probe 10 into the oral cavity of the subject such as a patient and attempts to bring the front face 21 of the light-emitting portion 10A into contact with the gum GU and tooth TO undergoing examination, the fingers of the user holding the gripping portion 10B will not readily touch the gum GU and tooth TO undergoing examination. In a case where the front face 21 of the light-emitting portion 10A is made to contact the target of the examination, the angle (direction) of light emission is easier to adjust so as to irradiate the tooth TO and gum GU with the measuring light beams B11, B21, B31, B41 and B51 perpendicularly in comparison with a case where such contact is not made.

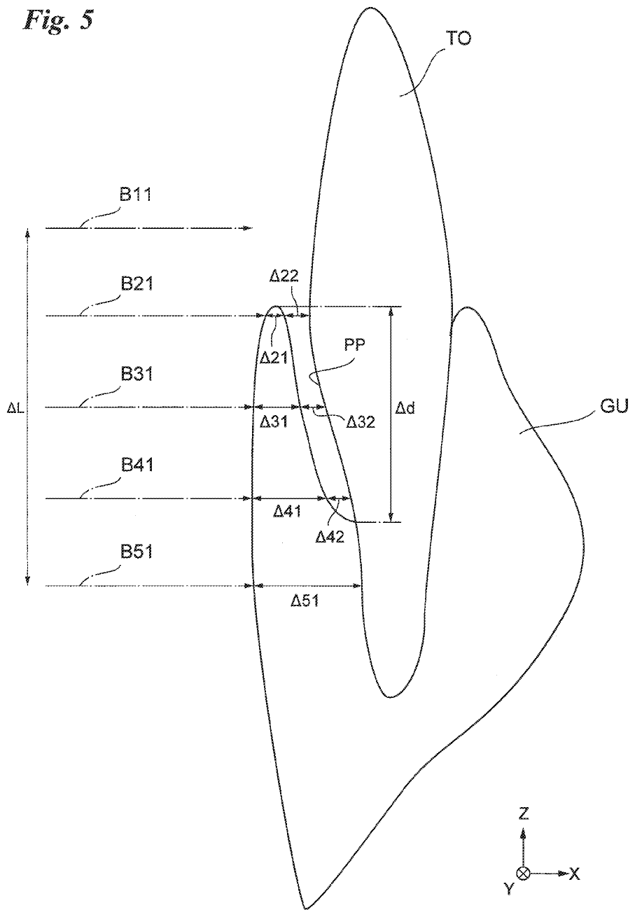

FIG. 5 illustrates the manner in which the gum GU and the tooth TO undergoing examination are irradiated with the measuring light beams B11, B21, B31, B41 and B51. FIG. 5 is enlarged as compared with FIG. 2.

In FIG. 5, the gum GU and tooth TO are seen from the side. The left side in FIG. 5 corresponds to one of either the outside or the inside of the body, and the right side corresponds to the other one of either the outside or the inside of the body.

The periodontal pocket PP is formed between the gum GU and tooth TO, as mentioned above. In the case of severe periodontal disease, the depth of the periodontal pocket PP is 6 mm or more. If deflection width ΔL of the measuring light beams B11 to B51 (deflection width of the measuring light beams B11 to B51 along the depth direction of the periodontal pocket PP) is 6 mm or more, therefore, whether the periodontal pocket PP exhibits sever periodontal disease can be determined. Accordingly, the selection of the crystal deflecting element 11 and the voltage applied thereto are decided in such a manner that the deflection width ΔL of the measuring light beams B11 to B51 will be 6 mm or more. Thus, enough deflection width to measure the depth of a periodontal pocket in a single scan is preferred.

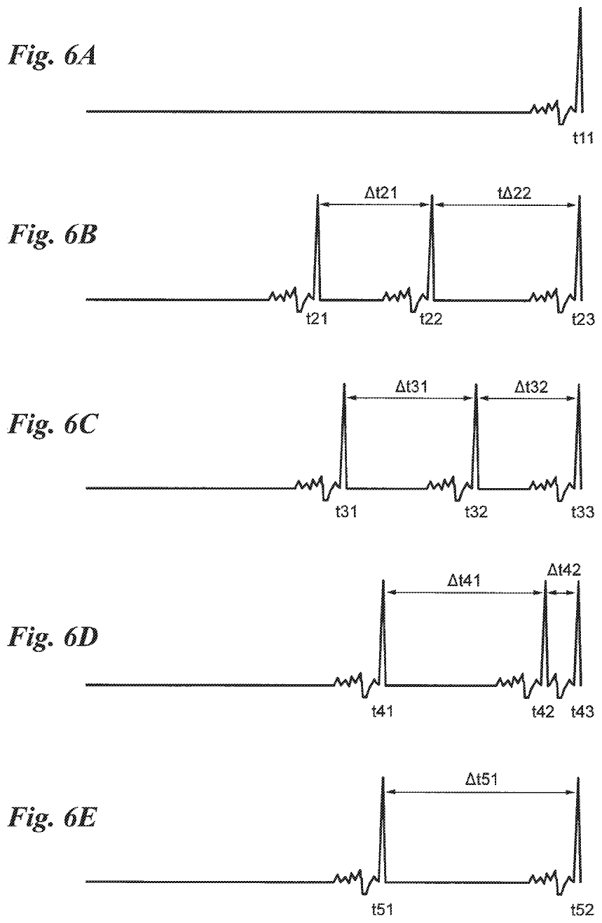

FIG. 6A to FIG. 6E are examples of interference signals.

FIG. 6A, FIG. 6B, FIG. 6C, FIG. 6D and FIG. 6E are examples of interference signals obtained based on the measuring light beams B11, B21, B31, B41 and B51, respectively.

The measuring light beam B11 directly irradiates the portion of the tooth TO where the gum GU is not present (see FIG. 5), and the intensity of the light reflected from the surface of the tooth TO rises. Based on the light reflected from the surface of the tooth TO, therefore, an interference signal is generated at time t11, as illustrated in FIG. 6A.

Since the measuring light beam B21 irradiates the upper end of the periodontal pocket PP (see FIG. 5), the intensity of the light reflected from the surface of the gum GU, from the boundary between the gum GU and the periodontal pocket PP, and from the surface of the gum GU, rises. As illustrated in FIG. 6B, therefore, interference signals are generated at times t21, t22 and t23 based on the light reflected from the surface of the gum GU, from the boundary between the gum GU and the periodontal pocket PP, and from the surface of the tooth TO, respectively. A time difference Δt21 from time t21 to time t22 indicates thickness Δ21 of the gum GU at the portion irradiated with the measuring light beam B21, and a time difference Δt22 from time t22 to time t23 indicates distance Δ22 across the gap (the distance across the space between the tooth TO and gum GU) of the periodontal pocket PP at the portion irradiated with measuring light beam B21.

Similarly, interference signals are generated at times t31, t32 and t33, as illustrated in FIG. 6C, based on the light reflected from the surface of the gum GU, from the boundary between the gum GU and the periodontal pocket PP, and from the surface of the tooth TO, respectively, owing to irradiation with the measuring light beam B31. A time difference Δt31 from time t31 to time t32 indicates thickness Δ31 of the gum GU at the portion irradiated with the measuring light beam B31, and a time difference Δt32 from time t32 to time t33 indicates distance Δ32 across the gap of the periodontal pocket PP at the portion irradiated with measuring light beam B31.

Similarly, interference signals are generated at times t41, t42 and t43, as illustrated in FIG. 6D, based on the light reflected from the surface of the gum GU, from the boundary between the gum GU and the periodontal pocket PP, and from the surface of the tooth TO, respectively, owing to irradiation with the measuring light beam B41. A time difference Δt41 from time t41 to time t42 indicates thickness Δ41 of the gum GU at the portion irradiated with the measuring light beam B41, and a time difference Δt42 from time t42 to time t43 indicates distance Δ42 across the gap of the periodontal pocket PP at the portion irradiated with measuring light beam B41.

No periodontal pocket PP has formed at the portion of the gum GU irradiated with the measuring light beam B51 (see FIG. 5). Based on the light reflected from the gum GU and from the surface of the tooth TO owing to irradiation with the measuring light beam B5, therefore, interference signals are generated at times t51 and t52, as illustrated in FIG. 6E. A time difference Δt51 from time t51 to time t52 indicates thickness Δ51 of the gum GU at the portion irradiated with the measuring light beam B51.

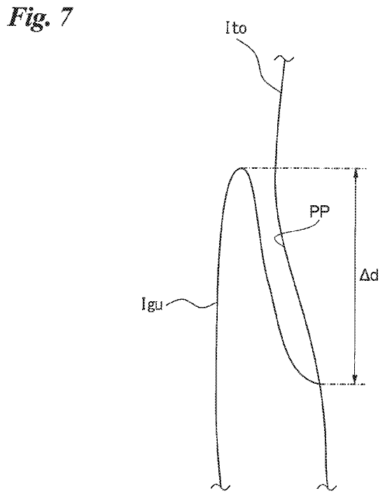

Optical tomographic images of the gum GU and tooth TO shown in FIG. 7 are generated by plotting the peak values of the interference signals of FIG. 6A to FIG. 6E.

FIG. 7 is an example of an optical tomographic image Igu of the gum GU and an optical tomographic image Ito of the tooth TO.

The optical tomographic image Igu of the gum GU and the optical tomographic image Ito of the tooth TO are displayed on the display screen of the display unit 6. By subjecting the optical tomographic image Igu of the gum GU and the optical tomographic image Ito of the tooth TO to contour extraction in the signal processing circuit 5, the depth Δd of the periodontal pocket PP is calculated in the signal processing circuit 5.

In this embodiment, the depth Δd of the periodontal pocket PP is calculated by generating the optical tomographic images Igu and Ito of the gum GU and tooth TO and extracting the contours of the generated optical tomographic images Igu and Ito. However, as will be described next, the depth Δd of the periodontal pocket PP may be calculated by computation without generating the optical tomographic images Igu and Ito (although the optical tomographic images Igu and Ito may just as well be generated).

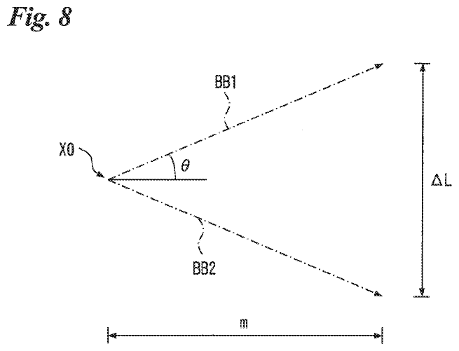

FIG. 8 illustrates measuring light beams BB1 and BB2 deflected by the crystal deflecting element 11.

It will be assumed that the measuring light beams BB1 and BB2 have the maximum deflection angle that result from the crystal deflecting element 11. If we let θ be the deflection angle of the measuring light beam BB1 or BB2, then the deflection width ΔL of the measuring light beams BB1 and BB2 obtained at a location spaced away a distance m along the light-emission direction from a reference point x0 prior to deflection will be ΔL=2 m·tan θ.

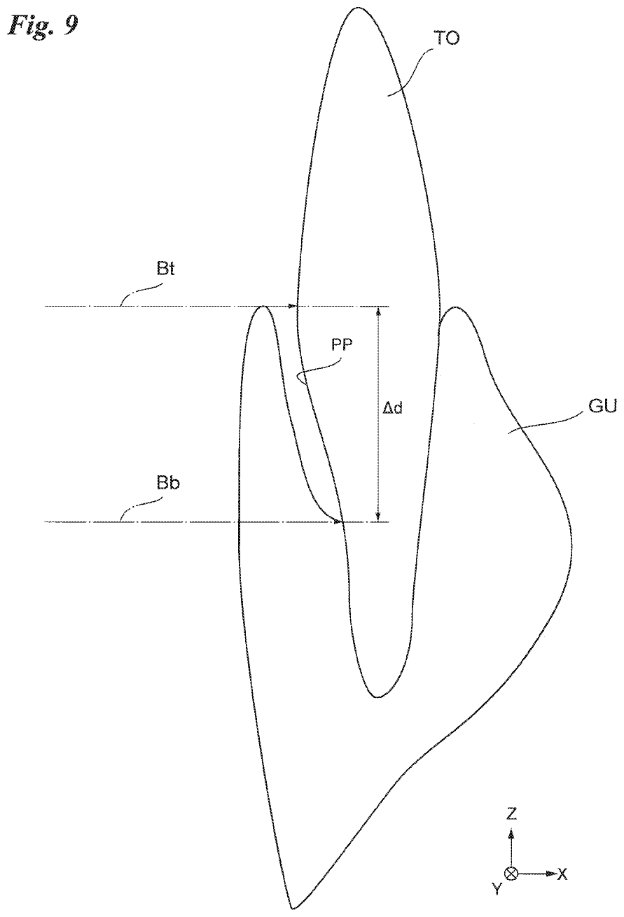

FIG. 9 illustrates the manner in which the gum GU and tooth TO undergoing examination are irradiated with measuring light beams Bt and Bb. FIG. 9 corresponds to FIG. 5.

The measuring light beam Bt irradiates a position corresponding to the upper end of the periodontal pocket PP, and the measuring light beam Bb irradiates a position corresponding to the lower end of the periodontal pocket PP. The distance from the position irradiated with the measuring light beam Bt to the position irradiated with the measuring light beam Bb corresponds to the depth Δd of the periodontal pocket pp.

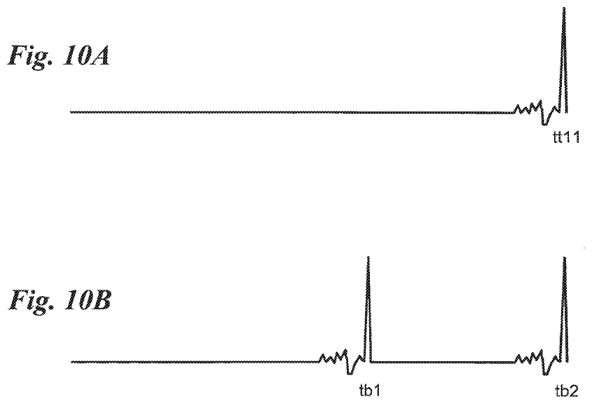

FIG. 10A is an example of an interference signal obtained based on the measuring light beam Bt, and FIG. 10B is an example of interference signals obtained based on the measuring light beam Bt and the measuring light beam Bb.

Since the measuring light beam Bt irradiates the tooth TO and not the gum GU, an interference signal is generated at a time tt1 owing to reflection from the tooth TO. Since the measuring light beam Bb irradiates the gum GU and the tooth TO, an interference signal is generated at a time tb1 owing to reflection from the gum GU and an interference signal is generated at a time tb2 owing to reflection from the tooth TO.

Accordingly, as described above with reference to FIG. 8, if we let T represent the length of time from that at which the measuring light beam BB1 having the maximum deflection angle is emitted until that at which the measuring light beam BB2 having the maximum deflection angle is emitted (let this length of time be one period), then T: ΔL=(tb2−tt1): Δd will hold. Therefore, the depth Δd of the periodontal pocket is calculated from Δd={ΔL·(tb2−tt1)}/T. Thus, the periodontal pocket Δd can be calculated even if the optical tomographic image Igu of the gum GU and the optical tomographic image Ito of the tooth TO are not always generated. It goes without saying that the calculation of the periodontal pocket Δd can be performed by the signal processing circuit 5.

In the embodiment set forth above, it is assumed that the deflection width from the measuring light beam B11 to B51 is enough to enable measurement of the depth Δd of the periodontal pocket in a single scan even in case of severe periodontal disease. However, in instances where there is not enough deflection width to enable measurement of the depth Δd of the periodontal pocket in a single scan, an arrangement may be adopted in which, by performing measurement multiple times at positions that differ in height (at least at two locations), data regarding the depth Δd of the periodontal pocket will be generated in the signal processing circuit (periodontal pocket data generator) based upon interference signals output from the photodiode 4.

For example, assume that the examination probe 10 can emit measuring light having a deflection width corresponding to the range from measuring light beam B11 to B31 (equal to the range from B31 to B51), which is illustrated in FIG. 5, by a single scan (measurement). First, assume that a first scan (measurement) by the examination probe 10 is carried out, at positions at which measuring light is capable of being emitted, over a range corresponding to the measuring light beams B11 to B31 illustrated in FIG. 5. In this case, optical tomographic images Igu and Ito of the upper half of gum GU and tooth TO shown in FIG. 5 are obtained from interference signals based on measuring light emitted over the range from measuring light beam B11 to B31, shown in FIG. 5, in the first scan. Next, the examination probe 10 is moved downward. Assume that, owing to a second scan performed at the position of the probe after such movement, measuring light from the examination probe 10 is emitted over the range from measuring light beam B31 to B51 shown in FIG. 5. In this case, optical tomographic images Igu and Ito of the lower half of gum GU and tooth TO shown in FIG. 5 are obtained from interference signals based on measuring light emitted over the range from measuring light beam B31 to B51, shown in FIG. 5, in the second scan. By subjecting the two optical tomographic images, which have been obtained by measurement performed at the positions of two points of different height, to combining processing in the signal processing circuit 5, the optical tomographic images of the gum GU and tooth TO shown in FIG. 5 are obtained. Needless to say, the optical tomographic images Igu and Ito of the upper half of gum GU and tooth TO and the optical tomographic images Igu and Ito of the lower half of gum GU and tooth TO are combined so as to be superimposed with regard to the overlapping portions thereof, and the connectivity of the optical tomographic images in the vertical direction is assured so as to obtain optical tomographic images that will be identical to the optical tomographic images Igu and Ito that would be obtained by a single scan.

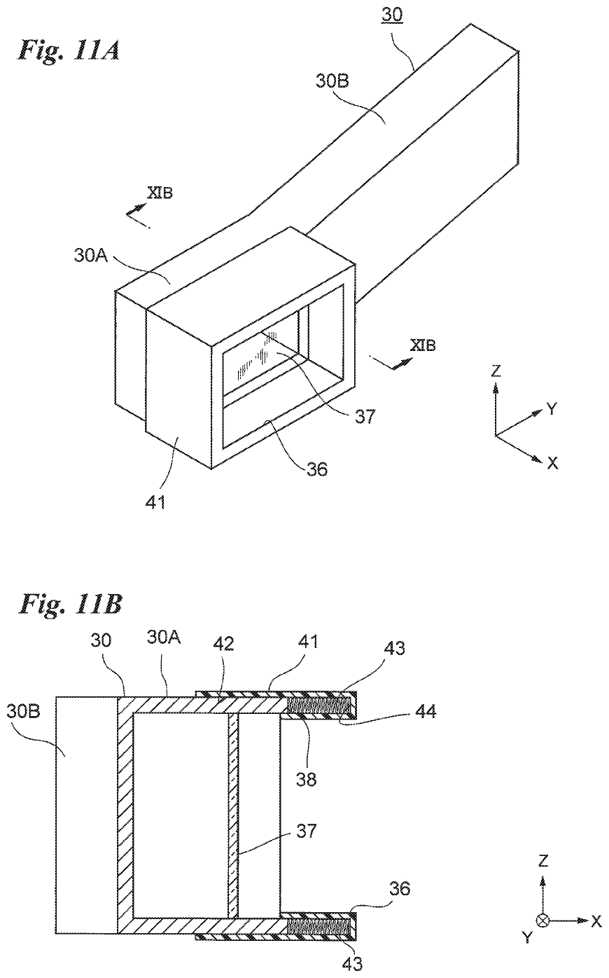

FIG. 11A and FIG. 11B illustrate a modification of the examination probe.

FIG. 11A, which corresponds to FIG. 4, is a perspective view of an examination probe 30 as seen from the front, and FIG. 11B is a sectional view taken along line XIB-XIB of FIG. 11A.

The examination probe 30 includes a light-emitting portion 30A and a gripping portion 30B.

The light-emitting portion 30A is a rectangular frame as seen from the front. Attached to the rectangular frame is a cover 41 freely slidable along the direction of emission of the measuring light emitted from the light-emitting portion 30A, as well as along the direction opposite thereto. The transparent plate 37 is fixed at a position inwardly of an opening 36, which is at the front of the cover 41, in the negative direction of the X-axis. The measuring light beams that have been rendered parallel are emitted from the opening 36 along the positive direction of the X-axis through the transparent plate 37, as mentioned above.

As illustrated in FIG. 11B, the interior of the cover 41 is formed to have a recess 42 shaped such that a front face 38 of the frame of the light-emitting portion 30A will fit therein. A compression spring 43 is secured between the front face 38 of the frame of light-emitting portion 30A and an inner wall 44 of the recess 42. When a force is applied to the cover 41 along the negative direction of the X-axis, a repulsive force acts along the positive direction of the X-axis owing to the compression spring 43. Thus the light-emitting portion 30A of the examination probe 30 shown in FIG. 11A and FIG. 11B is freely deformable such that the light-emitting portion 30A is deformed in a case where a force is applied to the light-emitting portion 30A along the negative direction of the X-axis, which is opposite the positive direction along the X-axis that is the direction in which the parallelized measuring light is emitted, and returns to the shape that prevailed prior to deformation in a case where the force applied to the light-emitting portion 30A is released. The portion of the cover 41 on the inside of the light-emitting portion 30A is shorter, along the negative direction of the X-axis, than the portion on the outside of the light-emitting portion 30A. Even if the cover 41 is moved along the negative direction of the X-axis, the transparent plate 37 will not obstruct movement of the cover 41 because the transparent plate 37 and the cover 41 do not come into contact.

In the embodiment shown in FIG. 11, owing to the fact that the cover 41 is subjected to a force in the direction opposite the direction of emission of the parallelized measuring light, the entire cover 41 is deformed in this opposite direction. That is, if the cover 41 is thought of as being a part of the light-emitting portion 30A, it is considered that the entire frame of the light-emitting portion 30A (examination probe 30) is deformed when a force is applied in the direction opposite the direction of emission of the parallelized measuring light. On the other hand, an arrangement may be adopted in which a cover is attached to a part of the upper portion and a part of the lower portion of the light-emitting portion 30A and the cover attached to these portions is deformed. Thus, the upper portion of the opening 36 of light-emitting portion 30A (where the “upper portion” refers to the upper portion in a case where the longitudinal direction of the examination probe 30 and the direction of emission of the measuring light are both taken as being horizontal) and the lower portion thereof (where the “lower portion” refers to the lower portion in a case where the longitudinal direction of the examination probe 30 and the direction of emission of the measuring light are both taken as being horizontal) are freely deformable such that they are deformed when a force is applied, in the direction (the negative direction along the X-axis) opposite the direction (the positive direction along the X-axis) in which the parallelized measuring light is emitted, across at least a portion (e.g., across a length greater than that of the tooth TO along the width direction) in the width direction [which is the positive direction along the Y-axis in FIG. 11A, the longitudinal direction of the examination probe 30 being taken as the width direction], and return to the shape that prevailed prior to deformation when the force applied is released.

Owing to the fact that portions of the light-emitting portion 30A are freely deformable, the front face of the light-emitting portion 30A (cover 41) can be brought into close contact with the gum GU and tooth TO of the subject.

With the examination probe 10 shown in FIG. 4, the longitudinal direction of the gripping portion 10B is taken as the direction (positive direction along the Y-axis) perpendicular to the direction (positive direction along the X-axis) of emission of the measuring light emitted from the opening 16. With the embodiment shown in FIG. 11A and FIG. 11B, however, the longitudinal direction of the gripping portion 30B is not the direction (positive direction along the Y-axis) perpendicular to the direction (positive direction along the X-axis) of emission of the measuring light emitted from the opening 36 but instead leans in a direction (the negative direction along the X-axis) opposite the direction (positive direction along the X-axis) of emission of the measuring light emitted from the opening 36. Thus, the gripping portion 30B leans in the direction opposite the direction of emission of the measuring light. When the gripping portion 30B is held, therefore, it is more difficult for the fingers holding the gripping portion 30B to touch the gum GU and tooth TO and it is easier for the front face of the light-emitting portion 30A to be brought into close contact with the gum GU and tooth TO.

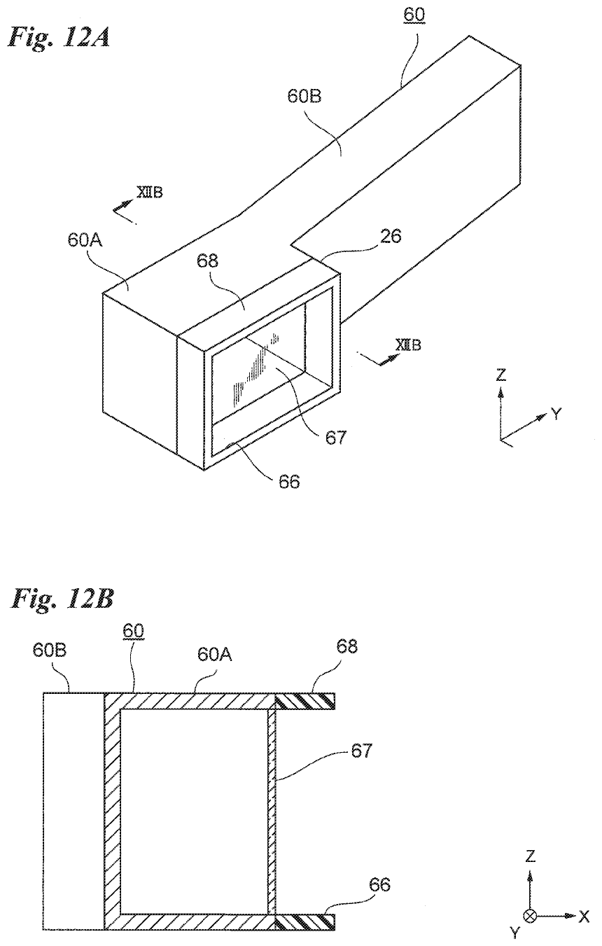

FIG. 12A and FIG. 12B show another example of an examination probe 60, in which FIG. 12A is a perspective view of the examination probe 60 and FIG. 12B a sectional view as seen along line XIIB-XIIB of FIG. 12A.

A light-emitting portion 60A is constituted by a frame the entirety of the front face of which is made of a freely deformable resin portion 68 such as rubber. The resin portion 68 also is freely deformable such that it is deformed in a case where a force is applied in the direction (the negative direction along the X-axis) opposite the direction (the positive direction along the X-axis) of emission of the parallelized measuring light, and returns to the shape that prevailed prior to deformation in a case where the applied force is released. A transparent plate 67 is fixed at a position inwardly of an opening 66, which is at the front of the resin portion 68, in the negative direction of the X-axis. As a result, deformation of the resin portion 68 is not restricted by the transparent plate 67.

In the examination probe 60 shown in FIG. 12A and FIG. 12B, parts of the upper and lower portions of the opening 66 of the light-emitting portion 60A may be made resin portions 68. Thus, the resin portion 68 is formed across at least a portion (e.g., across a length greater than that of the tooth TO along the width direction) in the width direction (Y-axis direction), and is freely deformable such that it is deformed when a force is applied in the direction (the negative direction along the X-axis) opposite the direction (the positive direction along the X-axis) in which the parallelized measuring light is emitted, and returns to the shape that prevailed prior to deformation when the force applied is released.

The front face of the light-emitting portion 60A can be brought into close contact with the gum GU and tooth TO also in the case where resin is utilized as portions of the light-emitting portion 60A.

In FIG. 11A and FIG. 11B, the compression spring 43 is utilized. Although a resin is utilized in FIG. 12A and FIG. 12B, it goes without saying that another material can be utilized if it is an elastic member which will be deformed when a force is applied and return to the shape that prevailed prior to deformation when the force is released.

Both the light-emitting portion 30A in FIG. 11A and FIG. 11B and the light-emitting portion 60A in FIG. 12A and FIG. 12B are rectangles as seen from the front but may just as well be circles. Even in the case of a light-emitting portion that is a circle as seen from the front, it can be so arranged that it will be deformed when a force is applied in the direction opposite the direction of emission of the measuring light and will return to the shape that prevailed prior to deformation when the force is released, in a manner similar to that described above. In a case where the front face of the light-emitting portion or the opening formed in the light-emitting portion is a circle, the upper side relative to the horizontal plane passing through the center of the circle is the upper portion of the light-emitting portion or of the opening, and the lower side relative to this horizontal plane is the lower portion of the light-emitting portion or of the opening.

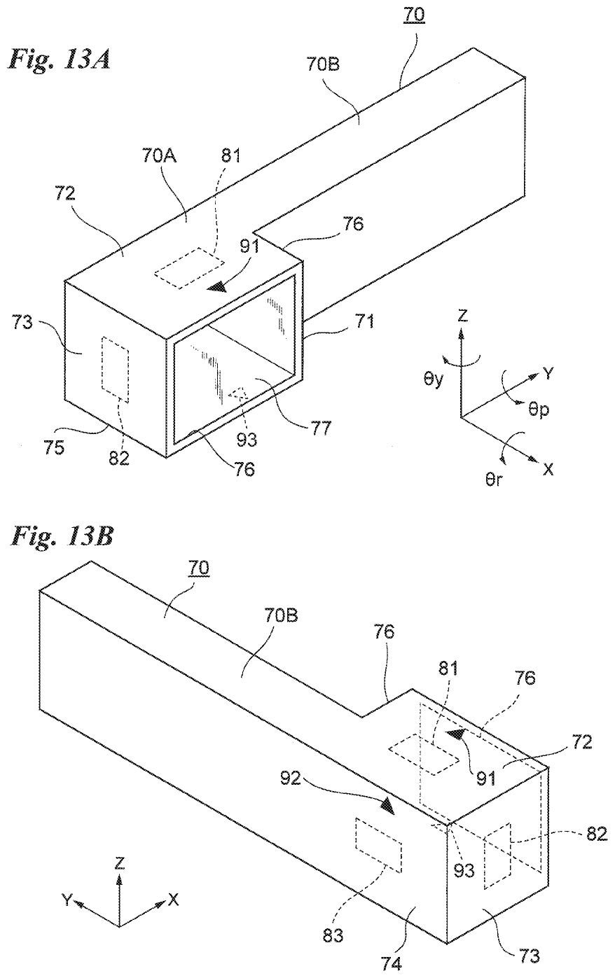

FIG. 13A and FIG. 13B illustrate another embodiment and are perspective views of an examination probe 70. FIG. 13A is a perspective view as seen from the front side, and FIG. 13B is a perspective view as seen from the back side.

The examination probe 70 includes a light-emitting portion 70A and a gripping portion 70B. A first angle sensor 81, a second angle sensor 82 and a third angle sensor 83 are embedded respectively in an upper plate 72, side plate 73 and back plate 74 of the light-emitting portion 70A. If we let a roll angle θr represent the angle about the X-axis, let a pitch angle θp represent the angle about the Y-axis and let a yaw angle θy represent the angle about the Z-axis, then the first angle sensor 81 will detect the yaw angle θy, the second angle sensor 82 will detect the pitch angle θp, and the third angle sensor 83 will detect the roll angle θr.

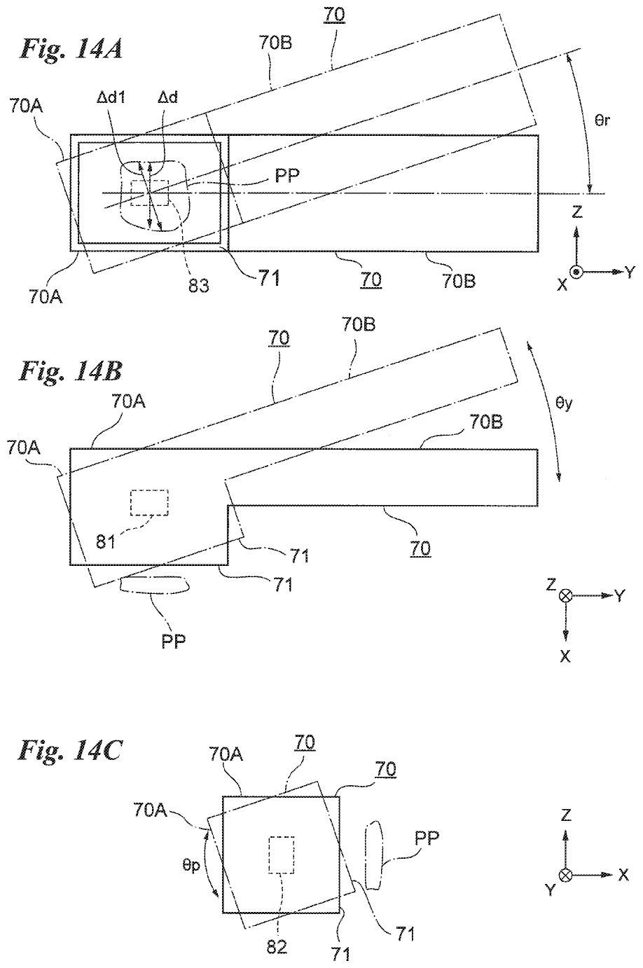

FIG. 14A illustrates the manner in which the roll angle θr is detected, FIG. 14B the manner in which the yaw angle θy is detected, and FIG. 14C the manner in which the pitch angle θp is detected.

FIG. 14A is a front view of the examination probe 70.

If the longitudinal direction of the examination probe 70 coincides with the direction of the Y-axis, as indicated by the solid line, the roll angle θr of the examination probe 70 will be 0 degrees. When the examination probe 70 is tilted about the X-axis, as indicated by the chain line, the roll angle θr is produced. If a front face 71 of the light-emitting portion 70A faces the gum GU and tooth TO in parallel fashion, interference signals are generated by measuring light reflected by the gum GU and tooth TO. As a result, the depth Δd of the periodontal pocket PP can be measured accurately. On the other hand, if the front face 71 of the light-emitting portion 70A of examination probe 70 does not face the gum GU and tooth TO in parallel fashion, there is a possibility that interference signals cannot be generated using the measuring light reflected by the gum GU and tooth TO. As a result, there is a possibility that the depth Δd of the periodontal pocket cannot be measured accurately, as for example an erroneous depth being measured as the depth Δd of the periodontal pocket. Further, if the vertical direction of the examination probe 70 and the direction of the depth Δd of the periodontal pocket do not coincide, there is a possibility that the depth Δd of the periodontal pocket cannot be measured accurately.

Since the roll angle θr is detected by the third angle sensor 83, the measurer is capable of grasping whether the vertical direction of the examination probe 70 and the direction of the depth Δd of the periodontal pocket coincide. It goes without saying that a signal indicating the roll angle θr detected by the third angle sensor 83 is input to the signal processing circuit 5 from the third angle sensor 83 and is displayed on the display screen of the display unit 6. Further, an arrangement may be adopted in which notification is given of an optimum roll angle by another method of notification, such as by issuing a sound, light (turning on a light-emitting diode, for example) or vibration.

The roll angle θr of the examination probe 70 with the front face 71 of the light-emitting portion 70A of the examination probe 70 facing the gum GU and tooth TO in parallel will differ in accordance with the angle of the face of the subject, or more specifically, the angle of the gum GU and tooth TO. Therefore, the roll angle θr of the examination probe 70 with the front face 71 of the light-emitting portion 70A of the examination probe 70 facing the gum GU and tooth TO in parallel may be calculated as by detecting the angle of inclination of the chair in which the subject is seated, by way of example. Alternatively, a specific roll angle θr of the examination probe 70 may be used as being the roll angle θr of the examination probe 70 with the front face 71 of the light-emitting portion 70A of the examination probe 70 facing the gum GU and tooth TO in parallel.

FIG. 14B is a top view of the examination probe 70.

If the longitudinal direction of the examination probe 70 coincides with the direction of the Y-axis, as indicated by the solid line, the yaw angle θy of the examination probe 70 will be 0 degrees. When the examination probe 70 is tilted about the Z-axis, as indicated by the chain line, the yaw angle θy is produced. If the front face 71 of the light-emitting portion 70A faces the gum GU and tooth TO in parallel fashion, interference signals are generated by measuring light reflected by the gum GU and tooth TO in a manner similar to that described above. As a result, the depth Δd of the periodontal pocket PP can be measured accurately. On the other hand, if the front face 71 of the light-emitting portion 70A of examination probe 70 does not face the gum GU and tooth TO in parallel fashion, there is a possibility that interference signals cannot be generated using the measuring light reflected by the gum GU and tooth TO. As a result, there is a possibility that the depth Δd of the periodontal pocket cannot be measured accurately, as for example an erroneous depth being measured as the depth Δd of the periodontal pocket.

Since the yaw angle θy is detected by the first angle sensor 81, it is possible to grasp whether the front face 71 of the light-emitting portion 70A of the examination probe 70 is facing the gum GU and tooth TO in parallel. It goes without saying that a signal indicating the yaw angle θy detected by the first angle sensor 81 also is input to the signal processing circuit 5 from the first angle sensor 81 and is displayed on the display screen of the display unit 6.

FIG. 14C is a left-side view of the examination probe 70.

If the measuring light emitted from the light-emitting portion 70A of the examination probe 70 coincides with the positive direction of the X-axis, as indicated by the solid line, the pitch angle θp of the examination probe 70 will be 0 degrees. When the examination probe 70 is tilted about the Y-axis, as indicated by the chain line, the pitch angle θp is produced. If the front face 71 of the light-emitting portion 70A faces the gum GU and tooth TO in parallel fashion, interference signals are generated by measuring light reflected by the gum GU and tooth TO in a manner similar to that described above. As a result, the depth Δd of the periodontal pocket PP can be measured accurately. On the other hand, if the front face 71 of the light-emitting portion 70A of examination probe 70 does not face the gum GU and tooth TO in parallel fashion, there is a possibility that interference signals cannot be generated using the measuring light reflected by the gum GU and tooth TO. As a result, there is a possibility that the depth Δd of the periodontal pocket cannot be measured accurately, as for example an erroneous depth being measured as the depth Δd of the periodontal pocket.

Since the pitch angle θp is detected by the second angle sensor 82, it is possible to grasp whether the front face 71 of the light-emitting portion 70A of the examination probe 70 is facing the gum GU and tooth TO in parallel. It goes without saying that a signal indicating the pitch angle θp detected by the second angle sensor 82 also is input to the signal processing circuit 5 from the second angle sensor 82 and is displayed on the display screen of the display unit 6.

An arrangement may be adopted in which, in a case also where the yaw angle θy and pitch angle θp are detected, notification is given of an optimum yaw angle θy and pitch angle θp by another method of notification, such as by issuing a sound, light (turning on a light-emitting diode, for example) or vibration.

The yaw angle θy and pitch angle θp of the examination probe 70 with the front face 71 of the light-emitting portion 70A of the examination probe 70 facing the gum GU and tooth TO in parallel will differ in accordance with the angle of the face of the subject, or more specifically, the angle of the gum GU and tooth TO. Therefore, as described above, the yaw angle θy and pitch angle θp of the examination probe 70 with the front face 71 of the light-emitting portion 70A of the examination probe 70 facing the gum GU and tooth TO in parallel may be calculated as by detecting the angle of inclination of the chair in which the subject is seated, by way of example. Alternatively, a specific yaw angle θy and pitch angle θp of the examination probe 70 may be used as being the yaw angle θy and pitch angle θp of the examination probe 70 with the front face 71 of the light-emitting portion 70A of the examination probe 70 facing the gum GU and tooth TO in parallel.

Returning to FIG. 13A and FIG. 13B, marks 91, 92 and 93 are provided on the upper plate 72, the back plate 74 and a lower plate 75 of the light-emitting portion 70A at positions corresponding to the position of emission of the measuring light emitted from the opening 76 of light-emitting portion 70A. More specifically, the positions at which the marks 91, 92 and 93 are provided correspond to the position, in terms of the longitudinal direction (width direction) of the examination probe 70, at which the measuring light is emitted. By looking at the marks 91, 92 and 93, the user such as a dentist can ascertain the position of emission of the measuring light even if the user cannot see the front face 71 of the light-emitting portion 70A. By looking at the marks 91, 92 and 93, the user such as a dentist can grasp the position of emission of the measuring light emitted from the light-emitting portion 70A of the examination probe 70 and can measure the depth of the periodontal pocket PP accurately.

Although the marks 91, 92 and 93 are formed on the upper plate 72, back plate 74 and lower plate 75 of the light-emitting portion 70A in FIG. 13A and FIG. 13B, they may be provided on any of the upper plate 72, back plate 74 and lower plate 75 or at any two locations. Further, an arrangement may be adopted in which a mark indicating the position of emission of the measuring light is provided on the front face 71 of the light-emitting portion 70A. A mark can thus provided on the exterior surface of the light-emitting portion 70A (the mark may be provided on the exterior surface except for the front face 71 of the light-emitting portion 70A). Furthermore, although the marks 91, 92 and 93 indicating the position corresponding to the position of emission of the measuring light are triangular in FIG. 13A and FIG. 13B, they may be other shapes, simple lines or simple recesses. Furthermore, an LED (Light-Emitting Diode) or the like may be provided at the position corresponding to the position of emission of the measuring light and may be used as a mark indicating the position of emission of the measuring light. In any case it will suffice if the position of emission of the measuring light can be ascertained.

Although the light-emitting portion 70A of examination probe 70 shown in FIG. 13A and FIG. 13B is a rectangular parallelepiped as seen from the front, it may just as well be a circular cylinder or hemisphere in shape as seen from the front. Even if the light-emitting portion 70A is cylindrical or hemispherical, the external surface of the light-emitting portion 70A with the exception of the front face 71 can be provided with the marks at positions corresponding to the position of emission of the measuring light emitted from the light-emitting portion.

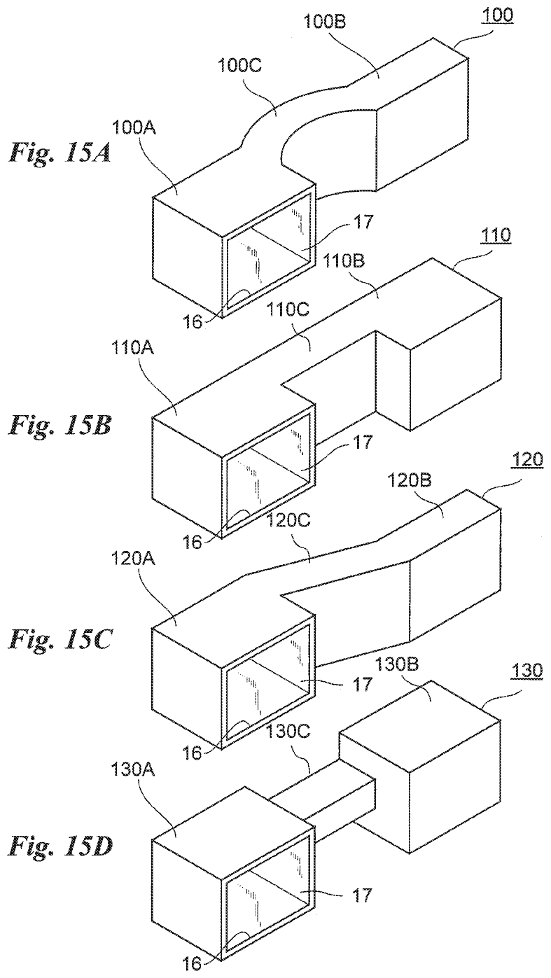

FIG. 15A to FIG. 15D, which illustrate examples of examination probes, are perspective views as seen from the front side. A neck portion is formed in all of the probes of FIG. 15A to FIG. 15D. Measuring light is emitted from the opening 16 via the transparent plate 17 in all of FIG. 15A to FIG. 15D.

With reference to FIG. 15A, an examination probe 100 includes a light-emitting portion 100A and a base-end portion 100B. The base-end portion 100B extends from the right-side face (one side face) of the light-emitting portion 100A via a neck portion 100C. The neck portion 100C, which curves in the direction opposite the direction of emission of the measuring light from the light-emitting portion 100A, protrudes in the direction opposite the direction of emission of the measuring light. The base-end portion 100B and neck portion 100C correspond to a gripping portion.

With reference to FIG. 15B, an examination probe 110 also includes a light-emitting portion 110A and a base-end portion 110B. The base-end portion 110B extends from the right-side face (one side face) of the light-emitting portion 110A via a neck portion 110C. The base-end portion 110B and neck portion 110C correspond to a gripping portion. The light-emitting portion 110A protrudes further than the neck portion 110C does along the direction of emission of the measuring light from the light-emitting portion 110A. Thus, even in a case where the light-emitting portion 110A protrudes further than a part of the gripping portion, not the entirety of the gripping portion, in the direction of emission of the measuring light, it is considered that the light-emitting portion 110A protrudes further than the gripping portion.

With reference to FIG. 15C, an examination probe 120 also includes a light-emitting portion 120A and a base-end portion 120B. The base-end portion 120B extends from the right-side face (one side face) of the light-emitting portion 120A via a neck portion 120C. The base-end portion 120B and neck portion 120C correspond to a gripping portion. The neck portion 120C is secured at one end thereof to the right-side face of the light-emitting portion 120A on the rear-end portion thereof, and extends toward the other end thereof in the direction of emission of the measuring light from the light-emitting portion 120A. The other end of the neck portion 120C is secured to the base-end portion 120B.

With reference to FIG. 15D, an examination probe 130 also includes a light-emitting portion 130A and a base-end portion 130B. The base-end portion 130B extends from the right-side face (one side face) of the light-emitting portion 130A via a neck portion 140C. The base-end portion 140B and neck portion 140C correspond to a gripping portion. The upper-end portion and lower-end portion of the neck portion 130C are cut away, one end of the neck portion 130C is secured to the central portion of the right-side face of the light-emitting portion 130A on the rear-end portion thereof, and the other end of the neck portion 130C is secured to the central portion of the left-side face of the light-emitting portion 130A on the rear-end portion thereof. In FIG. 15D, the neck portion 130C has both its upper- and lower-end portions cut away. However, either one of the upper-end portion and lower-end portion may be cut away.

As illustrated in FIG. 15A to FIG. 15D, the base-end portions 100B, 110B, 120B and 130B may extend from the light-emitting portion 100A, 110A, 120A or 130A via the neck portion 100C, 110C, 120C or 130C.

In FIG. 15A to FIG. 15D, the light-emitting portions 100A, 110A, 120A and 130A have the shape of a parallelepiped but may just as well be cylindrical or hemispherical. The opening 16 or the front face of the light-emitting portions 100A, 110A, 120A and 130A may be a square, a square, a rectangle, a circle, a rectangle whose side in the vertical direction is shorter than its side in the longitudinal direction, or an ellipse the longitudinal direction of which is the major axis and the vertical direction of which is the minor axis. The front face of the light-emitting portions 100A, 110A, 120A and 130A may be a circle or an ellipse and the opening 16 may be a rectangle, the front face of the light-emitting portions 100A, 110A, 120A and 130A may be a rectangle and the opening 16 may be a circle or an ellipse. Furthermore, the neck portions 100C, 110C, 120C and 130C need not necessarily be provided between the light-emitting portions 100A, 110A, 120A and 130A and the base-end portions 100B, 110B, 120B and 130B, respectively. It will suffice if the straight line along the longitudinal direction of the examination probe and the straight line along the direction of emission of the measuring light emitted from the light-emitting portions 100A, 110A, 120A and 130A (the straight line along the direction of the measuring light prior to its deflection by the crystal deflecting element) are non-parallel. Further, the straight line along the longitudinal direction of the examination probe and the straight line along the direction of emission of the measuring light emitted from the light-emitting portions 100A, 110A, 120A and 130A may be substantially orthogonal, and the straight line along the longitudinal direction of the examination probe and a plane parallel to the direction of the deflection width of the measuring light emitted from the light-emitting portions 100A, 110A, 120A and 130A may be substantially orthogonal.

1. A periodontal pocket examination apparatus comprising:
an optical divider for splitting low-interference light into measuring light and reference light;
a crystal deflecting device on which the measuring light split off by said optical divider is incident for deflecting the incident measuring light in a specific direction in accordance with an applied voltage and for emitting the measuring light deflected;
a parallelizing device for aligning into parallel light the measuring light emitted from said crystal deflecting device;
a photodetector for detecting reflected light and outputting an interference signal, the reflected light being reflected measuring light which is reflected from a gum or tooth owing to irradiation of the gum or tooth with the measuring light aligned parallel by said parallelizing device and reflected reference light which is split off by said optical divider and reflected by a reference surface;
periodontal pocket data generator for generating data regarding depth of a periodontal pocket based on the interference signal output from said photodetector; and
an examination probe including said crystal deflecting device, said parallelizing device and a gripping portion, the gripping portion extends from one side face of a light-emitter for emitting from an opening the measuring light aligned parallel by said parallelizing device, said light-emitter portion protruding further than said gripping portion does in the direction of emission of the measuring light.

2. A periodontal pocket examination apparatus according to claim 1, wherein the light-emitter of said examination probe is adapted so as to be freely deformable such that the light-emitter of said examination probe is deformed in a case where a force is applied to the light-emitter of said examination probe in the direction opposite the direction of emission of the measuring light, and returns to the shape thereof that prevailed prior to deformation in a case where the force applied to the light-emitter of said examination probe is released.

3. A periodontal pocket examination apparatus according to claim 2, wherein said examination probe is freely deformable such that the light-emitter of said examination probe, at an upper portion and lower portion of the opening of the light-emitter, is deformed in a case where a force is applied, over at least a portion in the width direction, in a direction opposite the direction of emission of the measuring light, and returns to the shape thereof that prevailed prior to deformation in a case where the force applied to the light-emitter of said examination probe is released.

4. A periodontal pocket examination apparatus according to claim 3, wherein at least a part of the upper portion and lower portion is constituted by an elastic member such that a front face of the light-emitter is capable of being brought into close contact with a gum or tooth.

5. A periodontal pocket examination apparatus according to claim 1, further comprising an angle sensor for detecting at least one among roll angle, pitch angle and yaw angle of the examination probe.

6. A periodontal pocket examination apparatus according to claim 1, wherein said crystal deflecting device deflects the incident measuring light in such a manner that deflection width of the measuring light emitted from the light-emitter of said examination probe is enough deflection width for measurement of depth of a periodontal pocket in a single scan.

7. A periodontal pocket examination apparatus according to claim 1, wherein said periodontal pocket data generator generates data regarding depth of a periodontal pocket, based on interference signals output from said photodetector by using said examination probe to perform measurement at least at two locations at positions that differ in height, in a case where the deflection width of the measuring light emitted from the light-emitter of said examination probe is less than enough deflection width for measurement of depth of a periodontal pocket in a single scan.

8. A periodontal pocket examination apparatus according to claim 7, further comprising optical tomographic image generator for generating at least two optical tomographic images, based on interference signals output from said photodetector by using said examination probe to perform measurement at least at two locations at positions that differ in height, in a case where the deflection width of the measuring light emitted from the light-emitting portion of said examination probe is less than enough deflection width for measurement of depth of a periodontal pocket in a single scan;
wherein said periodontal pocket data generator generates data regarding depth of a periodontal pocket by combining and processing at least two optical tomographic images generated by the optical tomographic image generating generator.

9. A periodontal pocket examination apparatus according to claim 1, wherein a position corresponding to a light-emission position of the measuring light emitted from the light-emitter is marked on the exterior of the light-emitter.

10. A periodontal pocket examination apparatus according to claim 1, wherein the opening of said examination probe or the front face of the light-emitter of said examination probe has the shape of a square, a circle, a rectangle whose side in the vertical direction is shorter than the side in the longitudinal direction, or an ellipse the longitudinal direction of which is the major axis and the vertical direction of which is the minor axis.

11. A periodontal pocket examination apparatus according to claim 1, wherein the gripping portion includes a base-end portion and a neck portion;
the base-end portion extends from one side face of the light-emitter of said examination probe via the neck portion; and
the neck portion curves in the direction opposite the direction of the light emission and protrudes in the direction opposite the direction of the light emission, or the light-emitter protrudes further than the neck portion does in the direction of the light emission, or one end of the neck portion is secured to a rear end of the light-emitter on one side face thereof and the other end of the neck portion protrudes further than the one end of the neck portion does in the direction of the light emission, or at least one of an upper-end portion and lower-end portion of the neck portion is cut away.

12. A periodontal pocket examination apparatus according to claim 1, wherein said examination probe is such that a straight line in the longitudinal direction along which the gripping portion of said examination probe extends and a straight line in the direction of the measuring light prior to deflection thereof by said crystal deflecting device are non-parallel.

13. A periodontal pocket examination apparatus according to claim 1, wherein said examination probe is such that a straight line in the longitudinal direction along which the gripping portion of said examination probe extends and a straight line in the direction of the measuring light prior to deflection thereof by said crystal deflecting device are orthogonal.

14. A periodontal pocket examination apparatus according to claim 1, further comprising a voltage circuit for impressing the applied voltage upon said crystal deflecting device;
wherein, on the one hand, when the applied voltage impressed by said voltage circuit is a positive voltage, said crystal deflecting device deflects the measuring light more in the specific direction in response to an increase in the positive voltage, and
when the applied voltage impressed by said voltage circuit is a negative voltage, said crystal deflecting device deflects the measuring light more in the direction opposite the specific direction in response to an increase in the negative voltage.

15. A periodontal pocket examination apparatus according to claim 1, wherein the light-emitter of said examination probe has a transparent plate; and
said transparent plate is fixed at a position inwardly of the opening of the light-emitter in a direction opposite the direction of the light emission.
